(19) United States
(12) Patent Application Publication
(10) Pub. No.: US 2010/0021530 A1
(43) Pub. Date: Jan. 28, 2010

(54) ENHANCED TRANS-KERATIN DRUG DELIVERY

(75) Inventor: Todd A. WEINFIELD, Redwood City, CA (US)

Correspondence Address: Brian S. Boyer, Ph.D., 493 Seaport Ct., Suite 105, Redwood City, CA, 94063, US

(73) Assignee: Innovation Biomedical Devices, Inc., Redwood City, CA (US)

(21) Appl. No.: 12/510,237

(22) Filed: Jul. 27, 2009

Related U.S. Application Data
(60) Provisional application No. 61/135,960, filed on Jul. 25, 2008.
(60) Provisional application No. 61/135,961, filed on Jul. 25, 2008.
(60) Provisional application No. 61/135,983, filed on Jul. 25, 2008.
(60) Provisional application No. 61/135,984, filed on Jul. 25, 2008.
(60) Provisional application No. 61/137,262, filed on Jul. 29, 2008.
(60) Provisional application No. 61/137,925, filed on Aug. 5, 2008.

(51) Int. Cl. A61K 31/4402; A61K 9/00; A61K 9/70; A61Q 3/00

(52) U.S. Cl. 424/449; 514/345; 424/400

(57) ABSTRACT

The teachings provided herein are directed to a system and method for delivering an anti-infective agent through the nail of a subject having a nail infection. The system can comprise a drug delivery mechanism comprising an anti-infective agent and a heating element and a holding mechanism for releasably attaching the drug delivery mechanism to the digit having the infected nail. The drug delivery mechanism does not comprise a nail-infection-agent-containing member or sponge for receiving and delivering the anti-infective agent to the infected nail. In addition, the holding mechanism comprises a substantially open structure that covers the infected nail and does not enclose the digit. The system facilitates an enhanced trans-keratin drug delivery of the anti-infective agent through the infected nail in a dark, warm, and moist environment that prevents the growth of fungi in the infected nail. The teachings also include a heatable bandage for treating an infected nail.

### CROSS-REFERENCE TO RELATED APPLICATIONS

This application claims the benefit of U.S. Provisional Application Nos. 61/135,960, filed Jul. 25, 2008; 61/135,961, filed Jul. 25, 2008; 61/135,983, filed Jul. 25, 2008; 61/135,984, filed Jul. 25, 2008; 61/137,262, Jul. 29, 2008; and, 61/137,925, Aug. 5, 2008; each of which is hereby incorporated herein by reference in its entirety.

### BACKGROUND

1. Field of the Invention

The teachings provided herein are directed to a system and method for delivering an anti-infective agent through the nail of a subject having a nail infection.

2. Description of the Related Art

It's unquestionable that a successful, safe, and non-invasive topical treatment for nail infections is a long-felt and unsolved need. This is an undeniable fact that is well-understood and accepted by those skilled in the art. The problem lies in transporting the anti-infective agents through the nail and into the nail bed, as well as other regions in and around the nail, to reach all sites and sources of the infection. The MEDLINE PLUS MEDICAL ENCYCLOPEDIA, for example, is a reputable extrinsic source for a teaching of the current state of the art and currently states the following:
“Fungal nail infections may be difficult to treat . . . it is common for the fungus to return. Over-the-counter creams and ointments generally do not help treat this condition[, and p]rescription antifungal medicines taken by mouth may help clear the fungus in about 50% of patients. However, such medicines can cause side effects or may interfere with other medications[, and s]ome of the oral medications used to treat fungal infections of the nail can harm the liver. In some cases, the health care provider may remove the nail. Nails grow slowly. Even if treatment is successful, a new, clearer nail may take up to a year to grow in. The fungal nail infection is cured by the growth of new, non-infected nails.”
See www.nlm.nih.gov/medlineplus/ency/article/001330.htm, viewed Jul. 24, 2009.”

Tinea unguium or onychomycosis (nail fungus), for example, has long been a medical challenge to cure. While there are topically applied reagents that effectively control fungal growth on skin (e.g., cyclopirox) getting the reagent to thoroughly contact the fungus throughout the nail and nail bed has long been the challenge. See Quintanar-Guerrero, et al., Universidad Nacional Autónoma de Mexico, Drug Dev Ind Pharm, 24(7): 685-90 (1998) (stating, the nail provides a seemingly impenetrable membrane protecting the fungus from outside elements).

One of skill will appreciate that research has long-focused on developing a topical reagent which both penetrates the nail and destroys the fungus. Unfortunately, the research has not produced a solution. Ciclopirox, terbinafine, azoles, and other topical antifungal agents have shown some positive results, but the cure rate is low, and at best around 20% after a year of treatment with ciclopirox, for example. Most cure rates are typically lower. This may be because the ciclopirox treatments target the fungal species tinea coporis, and much of the challenge is in developing a treatment of the 119 known strands of tinea. As such, the problem of developing a successful, safe, and non-invasive topical treatment for nail infections clearly remains.

Accordingly, those skilled in the art of treating nail infections, and the patients suffering such nail infections, will appreciate a successful, safe, and non-invasive topical treatment for nail infections. The present teachings provide such a method of treatment that (i) is topical and safe; (ii) does not require oral or systemic administration of drugs; (iii) is safer for patients that may be intolerant to systemic drug delivery; (iv) is several times faster than existing topical treatments; (v) does not require removal of the nail; and, as such, (vi) does not require the patient to do without the presence of a nail for the year or so required to grow a new nail. These are examples of the advantages that will be realized in the art by the teachings provided herein.

### SUMMARY

The teachings provided herein are directed to a system and method for delivering an anti-infective agent through the nail of a subject having a nail infection such as, for example, onychomycosis.

In some embodiments, the teachings are directed to a system for treating an infected nail. The system can comprise a drug delivery mechanism comprising an anti-infective agent and a heating element. The heating element can have a surface adapted for a direct contact with the anti-infective agent as administered directly to a dorsal surface of an infected nail of a digit of a subject. In these embodiments, the system can also comprise a holding mechanism for releasably attaching the drug delivery mechanism to the digit having the infected nail and maintaining the direct contact between the heating element and the anti-infective agent as administered directly to the dorsal surface of the infected nail. Moreover, in these embodiments, the drug delivery mechanism does not comprise a nail-infection-agent-containing member or sponge for receiving and delivering the anti-infective agent to the infected nail. In addition, the holding mechanism comprises a substantially open structure that covers the infected nail and does not enclose the digit. And, in these embodiments, the system facilitates an enhanced trans-keratin drug delivery of the anti-infective agent through the infected nail in a dark, warm, and moist environment that prevents the growth of fungi in the infected nail.

In some embodiments, the holding mechanism can promote a convection of heat in the direction of the nail bed. The promotion of the convection of can facilitate an enhanced trans-keratin drug delivery from the dorsal surface of the nail to the nail bed. While not intending to be bound by any theory or mechanism of action, the holding mechanism can provide substantially more heat insulation over the dorsal surface of the nail than a plantar or palmar surface of the digit to promote convection of heat toward the nail bed of the digit.

In some embodiments, the surface tension between the surface of the heater and the anti-infective agent can be preselected to further facilitate the transport of drug through the infected nail. In these embodiments, for example, the surface of the heater can be adapted for a direct contact with the anti-infective agent and comprise a non-polar material having a contact angle of over 90 degrees with the anti-infective agent, for example, to avoid a spreading or wetting of the anti-infective agent on the surface of heater.

The application of heat to the anti-infective agent facilitates the transport of the anti-infective agent through the infected nail. In some embodiments, the heating element maintains a temperature of about 45° C. at the dorsal surface of the nail. And, in some embodiments, the temperature gradient across the digit having the infected nail ranges from about 45° C. at the dorsal surface of the nail to about 37° C. or less at the plantar or palmar surface of the digit.

The chemistry of the anti-infective agent can be selected to facilitate the transport of the anti-infective agent through the infected nail. In some embodiments, the anti-infective agent comprises a keratolytic agent selected from the group consisting of lactic acid, allantoin, zinc pyrithione, sulfur, rosorcinol, undecylenic acid, and combinations thereof. In some embodiments, the anti-infective agent comprises an antifungal agent selected from the group consisting of clotrimazole, ketoconazole, miconazole, butenafine, econazole, lotrisone, naftifine, nystatin, oxiconazole, sulconazole, terbinafine, tolnaftate, sertaconazole, ertaczo, undecylenic acid, and combinations thereof. And, in some embodiments, the anti-infective agent can comprise a multi-functional component that functions as an antifungal, antibacterial, surfactant, keratolytic, and drying agent. In some embodiments, for example, the anti-infective agent comprises a component selected from the group consisting of hydrogen peroxide, poly(iminoimido carbonyl iminoimido carbony liminohexamethylenehydrochloride), bis(hydrogenated tallow alkyl) dimethyl quarternary chlorides, bis(hydrogenated alkyl methyl amines) hydrochloride, and combinations thereof. Moreover, in some embodiments, the anti-infective agent is selected from the group consisting of 1-bromo-3-chloro-5-dimethylhydantoin, 1,3-dichloro-5-dimethylhydantoin, 1,3-dichloro-5-ethyl-5-methylhydantoin, potassium peroxymonosulfate, and sodium dichloro-s-triazinetrione dehydrate.

The anti-infective agent can also comprise a surfactant to assist in controlling the surface tension between the keratin of the nail and the anti-infective agent delivery medium. In some embodiments, the surface tension is lowered using the surfactant. The surfactant can be, for example, anionic, cationic, amphoteric, or even non-ionic, wherein the surfactant can be selected to complement any delivery medium of the anti-infective agent.

The teachings are also directed to a method of treating an infected nail of a subject. In some embodiments, the methods can comprise obtaining a system described above, for example, and directly applying the anti-infective agent to the dorsal surface of the infected nail in the absence of a nail-infection-agent-containing member or sponge for receiving and delivering the anti-infective agent to the infected nail. The methods can further comprise releasably attaching the holding mechanism to the digit having the infected nail, wherein the releasably attaching includes directly contacting the anti-infective agent applied to the dorsal surface of the infected nail with the surface of the heating element. Heat is applied to the anti-infective agent contact is maintained between the anti-infective agent and the heating element to heat the anti-infective agent for a therapeutically effective treatment time.

As described above, the application of heat facilitates the transport of the anti-infective agent through the infected nail. As such, in some embodiments, the heating element maintains a temperature of up to about 45° C. at the dorsal surface of the nail. And, in some embodiments, the temperature gradient across the infected nail ranges from up to about 45° C. at the dorsal surface of the nail to about 37° C. or less at the plantar or palmar surface of the digit.

In some embodiments, the above-described chemistries can be used. However in some embodiments, the applying of the anti-infective agent to the dorsal surface of the infected nail comprises first applying a keratolytic agent without the application of heat as a pretreatment and then applying an anti-infective agent with heat. In these embodiments, for example, the keratolytic agent can be undecylenic acid, and the anti-infective agent can be terbinafine or clotrimazole.

The teachings also describe an apparatus for treating an infected nail. In these embodiments, the apparatus can comprise a heating element having a surface adapted for a direct contact with an anti-infective agent as administered directly to a dorsal surface of an infected nail of a digit of a subject. The surface can be adapted for any of a variety of functional purposes such as, for example, to provide additional comfort, or for inhibiting a leakage of the anti-infective agent from the apparatus. The apparatus can also comprise a holding mechanism for releasably attaching the heating element to the digit having the infected nail and maintaining the direct contact between the heating element and the anti-infective agent as administered directly to the dorsal surface of the infected nail. In these embodiments, however, the apparatus does not comprise a nail-infection-agent-containing member or sponge for receiving and delivering the anti-infective agent to the infected nail; the holding mechanism comprises a substantially open structure that covers the infected nail and does not enclose the digit; and, the heating element functions to heat the anti-infective agent to a temperature greater than about 40° C. and facilitate an enhanced trans-keratin drug delivery of the anti-infective agent through the infected nail in a dark, warm, and moist environment that prevents the growth of fungi in the infected nail.

The teachings are also directed to components that can be used for treating an infected nail using the teachings provided herein. In some embodiments, the teachings are directed to a heatable bandage for treating an infected nail. The heatable bandage can comprise, for example, a conformable sheet of material having an adhesive side and an external side. The sheet of material can be designed, for example, to hold a heating element against an anti-infective agent applied to a dorsal surface of an infected nail. In these embodiments, the heatable bandage can contain an electrical connection for operably connecting a power source to the heating element. The adhesive side of the sheet of material can be designed to adhere to epidermal tissues of a digit that surround an infected nail. The heating element can be designed to heat the anti-infective agent to a temperature greater than about 40° C. and facilitate an enhanced trans-keratin drug delivery of the anti-infective agent through the infected nail in a dark, warm, and moist environment that prevents the growth of fungi in the infected nail. In some embodiments, the system can include the heatable bandage, a power source, and an anti-infective agent.

### BRIEF DESCRIPTION OF THE FIGURES

FIGS. 1A and 1B illustrate the system for treating a nail, according to some embodiments.

FIGS. 2A and 2B illustrate the anatomy of a nail of the first digit of a foot, according to some embodiments.

FIG. 3 illustrates an apparatus that directly applies an anti-infective agent to the dorsal surface of an infected nail in the absence of a nail-infection-agent-containing member or sponge, according to some embodiments.

FIGS. 4A-4C illustrate a heatable bandage for treating an infected nail, according to some embodiments.

FIGS. 5A and 5B show the results of a treatment using an anti-infective agent retained by a sponge, heated, and applied to the dorsal surface of an infected nail, according to some embodiments.

FIG. 6 shows the successful treatment of an infected nail using the teachings described herein, according to some embodiments.

### DETAILED DESCRIPTION OF THE INVENTION

The teachings provided herein are directed to a system and method for delivering an anti-infective agent through the nail of a subject having a nail infection such as, for example, onychomycosis.

The treatment of an infected nail requires administering an agent to a subject having an infected nail. The terms “administration” or “administering” refer to a method of incorporating a composition into the cells or tissues of a subject, either in vivo or ex vivo to diagnose, prevent, treat, or ameliorate a symptom of a disease. In one example, a compound can be administered directly to the affected tissue of a subject. In another example, a compound can be administered to a subject by combining the compound with cell tissue from the subject ex vivo for purposes that include, but are not limited to, assays for determining utility and efficacy of a composition. When the compound is incorporated on the subject in combination with one or active agents, the terms “administration” or “administering” can include sequential or concurrent incorporation of the compound with the other agents such as, for example, any agent described above. A pharmaceutical composition of the invention is formulated to be compatible with its intended route of administration.

An “effective amount” of a compound of the invention can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount may need to be administered in an amount sufficient to result in amelioration of one or more symptoms of a disorder, prevention of the advancement of a disorder, or regression of a disorder. In one example, a therapeutically effective amount preferably refers to the amount of a therapeutic agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. The term “treating” refers to the administering one or more therapeutic or prophylactic agents taught herein.

A “prophylactically effective amount” refers to an amount that is effective at the dosages and periods of time necessary to achieve a desired prophylactic result such as, preventing or inhibiting the severity of condition. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

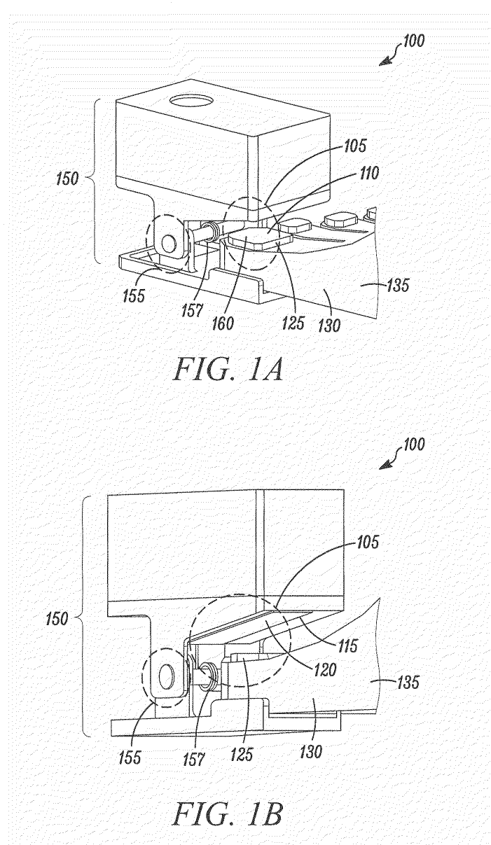

In some embodiments, the teachings are directed to a system for treating an infected nail. FIGS. 1A and 1B illustrate the system for treating a nail, according to some embodiments. The system 100 can comprise a drug delivery mechanism 105 comprising an anti-infective agent 110 and a heating element 115. The heating element 115 can have a surface 120 adapted for a direct contact with the anti-infective agent 110 as administered directly to a dorsal surface 160 of an infected nail 125 of a digit 130 of a foot 135 a subject.

In these embodiments, the system 100 also comprises a holding mechanism 150 for releasably attaching the drug delivery mechanism 105 to the digit 130 having the infected nail 125. Tensioner mechanism 155 includes spring 157 for maintaining the direct contact between the heating element 115 and the anti-infective agent 110 as administered directly to the dorsal surface 160 of the infected nail 125. Moreover, in these embodiments, the drug delivery mechanism 105 does not comprise a nail-infection-agent-containing member or sponge for receiving and delivering the anti-infective agent 110 to the infected nail 125. In addition, the holding mechanism 150 comprises a substantially open structure that covers the infected nail 125 and does not enclose the digit 130. And, in these embodiments, the system 100 facilitates an enhanced trans-keratin drug delivery of the anti-infective agent 110 through the infected nail 125 in a dark, warm, and moist environment that prevents the growth of fungi in the infected nail 125. It should be appreciated that system 100 can be designed with a custom fit for a particular digit, as a generic form for a variety of digit shapes and sizes, or for releasably attaching to one or more digits in a single application, either as a custom fit or generic form.

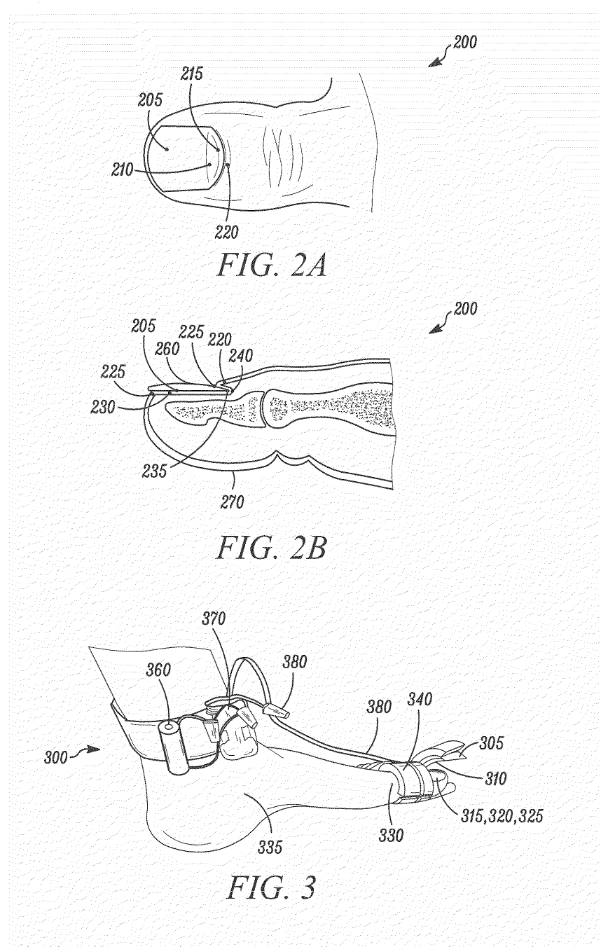

FIGS. 2A and 2B illustrate the anatomy of a nail of the first digit of a foot, according to some embodiments. FIG. 2A shows a top-view of digit 200 having nail plate 205, lunula 210, cuticle 215, and proximal nail fold 220. FIG. 2B shows a cross-sectional view of digit 200. The cross-sectional view further shows hyponychium 225, nail bed 230, nail root 235, and nail matrix 240. The dorsal surface 260 of the nail plate 205, and plantar surface 270 of the digit 200 (palmar surface in the case of a finger) are also shown. In some embodiments, the holding mechanism 150 can promote a convection of heat in the direction of the nail bed 230. The promotion of the convection of heat can facilitate an enhanced trans-keratin drug delivery from the dorsal surface 260 of the nail plate 205 to the nail bed 230. While not intending to be bound by any theory or mechanism of action, the holding mechanism 150 can provide substantially more heat insulation over the dorsal surface 260 of the nail plate 205 than a plantar surface 270 (or palmar surface) of the digit 200 to promote convection of heat toward the nail bed 230 of the digit 200.

The application of heat to the anti-infective agent facilitates the transport of the anti-infective agent through the infected nail. In some embodiments, the heating element maintains a temperature of about 45° C. at the dorsal surface of the nail. And, in some embodiments, the temperature gradient across the digit having the infected nail ranges from about 45° C. at the dorsal surface of the nail to about 37° C. or less at the plantar or palmar surface of the digit. It should be appreciated that the heating element can maintain any temperature considered by one of skill to be safe to the subject receiving the treatment. However, it should also be appreciated that, in some embodiments, the temperature can be limited to meet any government regulation regarding the application of heat to a tissue of a subject. For example, in some embodiments, the temperature applied to the dorsal surface of the nail can range from about 39° C. to about 50° C., from about 40° C. to about 49° C., from about 41° C. to about 48° C., from about 42° C. to about 47° C., from about 43° C. to about 46° C., about 45° C., or any range therein.

One of skill can select the safe amount of time in which a predetermined amount of heat can be applied to the nail. The local heat tolerance of human skin, for example, is known. It has been investigated in the art through the use of a thermostat-controlled heat-probe of 1 cm² contact area. See http://www.find-health-articles.com/rec_pub_{—}6869707-local-thermal-stress-tolerance-human-skin.htm, viewed Jul. 24, 2009. In some tests, 43° C. was the highest skin temperature which could be tolerated by a subject for about 8 hours with no restricted blood flow. The safe time of heat exposure has been observed to decrease dramatically with an increase in temperature of 1° C., reducing the time of heat tolerance to less than half in some tests. Applying heat in the amount of 44° C., for example, has been seen to produce strong thermal injury of the skin within 8 hours. The tolerable heat exposure time without causing thermal injury is shorter with restricted blood flow.

The anti-infective agent can be applied to the dorsal surface of the nail, and heat can be administered in direct contact with the anti-infective agent in brief cycles. The selection of the temperature and duration can vary, and one of skill can determine the best temperature and duration for a given subject and given anti-infective agent. The heat can be applied, for example, in time increments that range from about 1 minute to about 60 minutes in duration, and the heat can likewise be withdrawn in time increments that range from about 1 minute to about 60 minutes in duration. In some embodiments, the heat can be applied at about 41° C. to about 46° C. for time increments of about 15 minutes to about 45 minutes, where each heat application is followed by a rest period where the heat is withdrawn for time increments of about 15 minutes to about 45 minutes. The number of cycles of heat application can range from a single cycle per day to multiple cycles per day. For example, the heat can be applied 1, 2, 3, 4, 5, 6, 7, 8, 9, 10 times per day or more, and the cycles per day can varied to accommodate lifestyle, according to any suitable treatment regime selected. The process is repeated daily until obtaining the desired response which, in many embodiments, is a clinical cure of the nail infection. The term “clinical cure” generally refers to the alleviation of symptoms for a substantial period of time which suggests that the treated infection was eliminated and any re-occurrence of an infection is separate and distinct from the treated infection.

In some embodiments, the treatments are applied for a time frame ranging from about 1 week to about 6 months, from about 2 weeks to about 4 months, from about 3 weeks to about 3 months, from about 1 month to about 2 months, or any range therein. The duration of treatment will, of course, depend upon a variety of factors including, but not limited to, type of infection, severity of infection, age of the patient, frequency of treatment, selection of chemistry, selection of temperature, and any combination thereof.

The teachings provided herein include directly contacting the heating element with the anti-infective as applied to a dorsal surface of an infected nail. As such, in these embodiments, the system does not comprise a nail-infection-agent-containing member or sponge for receiving and delivering the anti-infective agent to the infected nail. The system has been designed to promote the transfer of anti-infective agent away from the surface of the heating element and toward the dorsal surface of the nail, through the nail, and into the nail bed. Accordingly, in some embodiments, the surface tension between the surface of the heater and the anti-infective agent can be preselected to further facilitate the transport of drug through the infected nail. In these embodiments, for example, the surface of the heater can be adapted for a direct contact with the anti-infective agent and comprise a non-polar material having a contact angle of over 90 degrees with the anti-infective agent, for example, to avoid a spreading or wetting of the anti-infective agent on the surface of heater.

The chemistry of the anti-infective agent can be selected to facilitate the transport of the anti-infective agent through the infected nail. The anti-infective agents can include, but are not limited to, agents having an antibacterial, antiviral, antifungal, antiprotozoal function, or a combination thereof. In some embodiments, the anti-infective agents include imazoles, allylamines and benzylamines, polyenes, synthetic antifungals, disinfectants, botanicals, and antibacterials.

Examples of imazoles include bifoconzole, butoconazole, clotrimazole, econazole, fluconazole, itraconazole, ketoconazole, miconazole, oxiconazole, saperconazole, sertaconazole, sulconazole, terconazole, tioconazole, voriconazole, and ioloconazole. Examples of allylamines and enzylamines include butenafine, naftifine, terbinafine. Examples of polyenes include amphotericin B, candidicin, filipin, fungimycin, and nystatin.

Examples of synthetic antifungal compounds include amorolfine (demethymorpholine), cicloprox olamine, haloprogen, clioquinol, tolnaftate, undecylenic acid, hydantoin, chlordantoin, pyrrolnitrin, ticlatone, triacetin, griseofulvin, and zinc pyrithione. Examples of disinfectants include copper sulfate, Gentian Violet, betadyne/povidone iodine, colloidal silver, and zinc.

Examples of botanicals include basil (oncimum basilicum), cassia (Cinnamomum aromaticum var. cassia), cedrus wood oil (Cedrus libani or Cedrus spp), chamomile (Chamaemelum nobile), citronella (Cymbopogon nardus), clove (Syzgium aromaticum), cumin (cuminum cyminum), fennel (Foeniculum vulgare), menthThe/Mint (MenthTHe×piperity/MethTHe spicata), tea tree Oil (Melaleuca alternfolia), tumeric leaf oil (curcumThe longa), and lemongrass oil (Cymbopogon citratus).

Examples of antibacterials include clindamycin, erythromycin, tetracycline, Metronidazole, sulfonamides, amoxicillin, penicillin, AMOXIL, demeclocycline, DECLOMYCIN, retapamulin, cephalosporins, cefoxitin, a cephamycin, fluoroquinolones tetracyclines, macrolides, aminoglycosides, lincosamindes, clindamycin, lincomycin, erythromycin, azithromycin, clarithyromycin, metronidazole, neomycin sulfate, aminoglycosides, polymyxin B, bacitracin zinc, pramoxine, sulfacetamide, sodium sulfacetamide lotion, doxycycline, minocycline, tinidazole, co-trimoxazole, cephamandole, ketoconazole, bacteriocins, microcins, lantibiotics, coliciniocins, probiotics, agrocin, alveicin, carnocin, colicin, curvaticin, divercin, enterocin, enterolysin, epidermin, erwiniocin, glycinecin, halocin, lactococin, lacticin, leucoccin, mesentericin, nisin, pediocin, plantaricin, sakacin, subtilin, sulfolobicin, vibriocin, warnerin, marigolds, chlorine, bromine, chloramphenicol, the cephalosporins, erythromycins, tetracyclines, aminoglycosides, 4-quinolones, and ciprofloxacin.

In some embodiments, the anti-infective agent comprises a keratolytic agent selected from the group consisting of lactic acid, allantoin, zinc pyrithione, sulfur, rosorcinol, undecylenic acid, and combinations thereof. In some embodiments, the keratolytic agent can include, but is not limited to, papain, salicylic acid, urea, lactic acid, allantoin, zincpyrithione, sulfur, resorcinol, magnesium sulfate, or combinations thereof.

In some embodiments, the anti-infective agent comprises an antifungal agent selected from the group consisting of clotrimazole, ketoconazole, miconazole, butenafine, econazole, lotrisone, naftifine, nystatin, oxiconazole, sulconazole, terbinafine, tolnaftate, sertaconazole, ertaczo, undecylenic acid, and combinations thereof. In some embodiments, the antifungal agent can include, but is not limited to, terbinafine hydrochloride, LAMISIL, amorolfine, demethymorpholine, LOCERYL, cicloprox olamine, clotrimazole, econzole, oxiconzole, triacetin, undecylenic acid, or a combination thereof.

Several compounds are contemplated for use as anti-infective agents with the present teachings. In some embodiments, the agents can include clotrimazole, ketoconazole, miconazole, butenafine, ciclopirox, econazole, lotrisone, naftifine, nystatin, oxiconazole, sulconazole, terbinafine, tolnaftate, sertaconazole, ertaczo, tea tree oil, VICKS VAPOR RUB, alcohol, vinegar, benzoic acid, iodochlorhydroxyquin, triacetin, undecylenic acid, bifonazole, butenafine, isoconazole nitrate, sodium propionate, sulconazole, griseofulvin, saliva, amphibian skin, invertebrates, lemon grass oil, sandalwood oil, cedrus oil, and clove bud oil.

In some embodiments, the agents can include Ocimum basilicum (basil), Cinnamomum aromaticum var Cassia (cinnamon), Cedrus libani (cedar of Lebanon), any Cedrus spp., Chamaemelum nobile (chamomile), Cymbopogon nardus (citronella), Syzygium aromaticum (clove & clove bud), Cumimim cyminum (cumin), Foeniculum vulgare (fennel), Melaleuca altemfolia (tea tree), Mentha×piperita (peppermint), Mentha spicata (spearmint), Curcuma longs (tumeric), Cymbopogon citrates (lemongrass), and Santalum album (sandalwood)

In some embodiments, the agents can include aliphatic nitrogen compounds, amide compounds, acylamino acid compounds, allylamine compounds, anilide compounds, benzanilide compounds, benzylamine compounds, furanilide compounds, sulforsunilide compounds, benzamide compounds, furamide compounds, phenylsulfamide compounds, sulfonamide compounds, valinamide compounds, antibiotic compounds, strobilurin compounds, aromatic compounds, benzimidazole compounds, benzimidazole precursor compounds, benzothiazole compounds, bridged diphenyl compounds, carbamate compounds, benzinaidazolylearbamme compounds, carbanilate compounds, conazole compounds, conazole compounds (imidazoles), conazole compounds (triazoles), copper compounds, dicarboximide compounds, dichlomphenyl dicarboximide compounds, phthalimide compounds, dinitrophenol compounds, dithiocarbamate compounds, cyclic dithiocarbamate compounds, polymeric dithiocarbamate compounds, imidazole compounds, inorganic compounds, mercury compounds, inorganic mercury compounds, organomercury compounds, morpholine compounds, organophosphorus compounds, organotin compounds, oxathiin compounds, oxazole compounds, and polyene compounds.

In some embodiments, the agents can include polysulfide compounds, pyrazole compounds, pyridine compounds, pyrimidine compounds, pyrrole compounds, quinoline compounds, quinone compounds, quinoxaline compounds, thiocarbamate compounds, thiazole compounds, thiophene compounds, triazine compounds, triazole compounds, urea compounds, amorolfine (dimethylmorpholine), bifonazole, butenafine, butoconazole, clioquinol, ciclopirox olamine, econazole, fluconazole, griseofulvin, haloprogen, iodochlorhydroxyquine, itracle, ketoconamle, miconazole, naftifine, oxiconazole, povidone-iodine sertaconazole, sulconazole, terbinafine, terconazole, tioconazole, tolnaftate, undecylenic acid and its salts (calcium, copper, and zinc), voriconazole, the sodium or zinc salts of proprionic acid, butylamine, cymoxanil, dodicin, Bodine, guazatine, iminoctadine, carpropamid, chloomforethan, cyflufenamid, diclocymet, ethaboxam, fenoxanil, flumetover, farametpyr, mundiproparand, penthiopyrad, prochloraz, quinazamid, silthiofinn, triforine, benalaxyl, benalaxyl-M, furalaxyl, metalaxyl, metalaxyl-M, pefzome, benalaxyl, benalaxyl-M, boscalid, carboxin, and fenhexamid.

In some embodiments, the agents can include metalaxyl, metalaxyl-M, metsulfovax, ofurace, oxadixyl, oxycarboxin, pyracarbolid, thifluzamide, tiadinil, benodanil, flutolanil, mebenil, mepronil, salicylanilide, lecloftalam, fenfuram, furalaxyl, furcarbanil, methfuroxam, flusulfamide, benzohydroxamic acid, fluopicolide, tioxymid, trichlamide, zarilamid, zoxamide, cyclafuramid, furmecyclox, dichlofluanid, tolylfluanid, amisulbrom, cyazofamid, benthiavalicarb, iprovalicarb, aureofungin, blasticidin-S, cycloheximide, griseofulvin, kasugamycin, mtamycin, polyoxins, polyoxorim, streptomycin, validamycin, azoxystrobin, dimoxystrobin, fluoxastrobin, kresoxim-methylmetominostmbin, orysastrobin, picoxystrobin, pymclostrobin, trifloxystrobin, biphenyl, chlorodinitronaphthalene, chloroneb, chlorothalonil, cresol, diclonin, hexachlorobenzene, pentachlorophenol, quintozene, sodium pentachlorophenoxide, tecnazene, benomyl, carbendazim, chlorfenazole, cypendazole, debacarb, fuberidazole, mecarbinzid, and rabenzazole.

In some embodiments, the agents can include thiabendazole, furophanate, thiophanate, thiophanate-methyl, bentaluron, chlobenthiazone, TCMTB, bithionol, dichlorophen, diphenylamine, benthiavalicarb, furophanate, iprovalicarb, propamocarb, thiophanme, thiophanate-methyl, benomyl, carbendazim, cypendazole, debacarb, mecarbiazid, diethofencarb, climbazole, imazalil, oxpoconazole, prochloraz, triflumizole, imidazole compounds, azaconazole, bromuconazole, cyproconazole, diclobutrazol, difenoconazole, diniconazole, diniconazole-M, epoxiconazole, etaconazole, fenbuconaole, fluquinconazole, flusilazole, flutriafol, furconazole, furconazole-cis, hexaconazole, imibenconazole, ipconazole, metconazole, myclobutarfil, penconazole, and propiconazole.

In some embodiments, the agents can include metconazole, myclobutanil, penconazole, propiconazole, prothioconazole, quinconazole, simeconazole, tebuconazole, tetraconazole, triadimefon, triadimenol, triticonazole, uniconazole, uniconazole-P, triazole compounds, Bordeaux mixture, Burgundy mixture, Cheshunt mixture, copper acetate, copper carbonate, basic, copper hydroxide, copper naphthenate, copper oleate, copper oxychloride, copper sulfate, copper sulfate, basic, copper zinc chromate, cufraneb, cuprobam, cuprous oxide, mancopper, oxine copper, famoxadone, fluoroimide, chlozolinate, dichlozoline, iprodione, isevaledicale, myclozolia, procymidone, vinclozolhi, captafol, captan, ditalimfos, folpet, thiochlorfenphim, bina-acryl, dinobuton, dinocap, dinocap-4, dinocap-6, dinocton, dinopenton, dinosulfon, dinoterbon, DNOC, azithiram, carbamorph, cufraneb, cuprobam, disulfiram, ferbam, metam, nabam, tecoram, thiram, ziram, dazomet, etem, milneb, mancopper, mancozeb, maneb, metimm, polycarbamate, propmeb, zineb, cyazofamid, fenamidone, fenapanil, glyodin, iprodione, isovaledione, pefurazoate, triazoxide, conazole compounds (imidazoles), potassium azide, potassium thiocyanate, sodium azide, and sulfur.

In some embodiments, the agents can include copper compounds, inorganic mercury compounds, mercuric chloride, mercuric oxide, mercurous chloride, (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-ptoluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercunarea, phenylmercury acetate, phenylmercury chloride, phenylmercury derivative of pyrocatechol, phenylmercury nitrate, phenylmercury salicylate, thiomersal, tolylmercury acetate, aldimorph, benzamorf, carbamorph, dimethomorph, dodemorph, fenpropimorph, flumorph, tridemorph, ampropylfos, dMinalbs, edifenphos, fosetyl, hexylthiofos, iprobenfos, phosdiphen, pyrazophos, tolclofosmethyl, triamiphos, and decafentin,

In some embodiments, the agents can include fentin, tributyltin oxide, carboxin, oxycarboxin, chlozolinate, dichlozoline, drazoxolon, famoxadone, hymexazol, metazoxolon, myclozolin, oxadixyl, vinclozolin, barium polysulfide, calcium polysulfide, potassium polysulfide, sodium polysulfide, furametpyr, penthiopyrad, boscalid, buthiobate, dipyritbione, fluazinam, fluopicolide, pyridinitril, pyrifenox, pymxycblor, pyroxylur, bupirimme, cyprodinil, diflumetorim, dimethirimol, otbirimol, fenarimol, ferimzone, mepanipyrim, nuarimol, pyrimethanil, triarimol, fenpiclonil, fludioxonil, fluoroimide, ethoxyquin, halacrinate, 8-hydroxyquinoline sulfate, quinacetol, quinoxyfen, benquinox, chloranil, dichlone, dithianon, chinomethionat, chlorquinox, thioquinox, ethaboxam, etridiazole, metsulfovax, octhilinone, thiabendazole, thiadifluor, thifluzamide, methasulfocarb, prothiocarb, ethaboxam, silthiofam, anilazine, amisulbrom, bitertanol, fluotrimazole, triazbutil, conazole compounds (triazoles), bentaluron, pencycuron, quinazamid, acibenzolar, acypetacs, ally] alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, plithalide, piperalin, probenazole, proquinazid, pyroquilon, sodium orthophenylphenoxide, spiroxamine, suhropen, thicyofen, tricyclazole, iodophor, silver, NYSTATIN, amphotericin B, griseofulvin, and zinc naphthenate.

In some embodiments, the agents can include bifoconazole, butoconazole, econazole, fluconazole, itraconazole, ketoconazole, miconazole, oxiconazole, saperconazole, sertaconazole, sulconazole, terconazole, tioconazole, voriconazole, ioloconazole, butenafine, naftifine, terbinafine, amphotericin B, candidicin, filipin, fungimycin, nystatin, amorolfine (demethymorpholine), cicloprox olamine, haloprogen, clioquinol, undecylenic acid, hydantoin, chlordantoin, pyrrolnitrin, salicylic acid, ticlatone, triacetin, griseofulvin, zinc pyrithione. copper sulfate, Gentian Violet, betadyne/povidone iodine, colloidal silver, zinc, and ANACOR AN2690.

In some embodiments, the anti-infective agent can comprise a multi-functional component that functions as an antifungal, antibacterial, surfactant, keratolytic, and drying agent. In some embodiments, for example, the anti-infective agent comprises a component selected from the group consisting of hydrogen peroxide, poly(iminoimido carbonyl iminoimido carbony liminohexamethylenehydrochloride), bis(hydrogenated tallow alkyl) dimethyl quarternary chlorides, bis(hydrogenated alkyl methyl amines) hydrochloride, and combinations thereof. Moreover, in some embodiments, the anti-infective agent is selected from the group consisting of 1-bromo-3-chloro-5-dimethylhydantoin, 1,3-dichloro-5-dimethylhydantoin, 1,3-dichloro-5-ethyl-5-methylhydantoin, potassium peroxymonosulfate, and sodium dichloro-s-triazinetrione dehydrate.

Other such agents can include clindamycin, erythromycin, tetracycline, metronidazole, sulfonamides, amoxicillin, penicillin, demeclocycline declomycin, retapamulin, cephalosporins, cefoxitin, a cephamycin, fluoroquinolones tetracyclines. macrolides aminoglycosides, lincosamindes, clindamycin and lincomycin, erythromycin, azithromycin, clarithyromycin, metronidazole, neomycin sulfate, aminoglycosides, polymyxin B, bacitracin zinc, pramoxine, sulfacetamide, sodium sulfacetamide lotion, minocycline, tinidazole, co-trimoxazole, cephamandole, ketoconazole, bacteriocins, microcins, lantibiotics, coliciniocins, probiotics, agrocin, alveicin, carnocin, colicin, curvaticin, divercin enterocin, enterolysin, epidermin, erwiniocin, glycinecin, halocin, lactococin, lacticin, leucoccin, mesentericin, nisin, pediocin, plantaricin, sakacin, subtilin, sulfolobicin, vibriocin, warnerin, marigolds, chlorine, bromine, chloramphenicol, the cephalosporins, erythromycins, tetracyclines, aminoglycosides, 4-quinolones, and ciprofloxacin.

Other such agents can include active chlorine (i.e., hypochlorites, chloramines, dichloroisocyanurate and trichloroisocyanurate, wet chlorine, chlorine dioxide etc.), active oxygen (peroxides, such as peracetic acid, potassium persulfate, sodium perborate, sodium percarbonate and urea perhydrate), iodine (iodpovidone (povidone-iodine, BETADINE), Lugol's solution, iodine tincture, iodinated nonionic surfactants), concentrated alcohols (mainly ethanol, 1-propanol, called also n-propanol and 2-propanol, called isopropanol and mixtures thereof; further, 2-phenoxyethanol and 1- and 2-phenoxypropanols are used), phenolic substances (such as phenol (also called “carbolic acid”), cresols (called “Lysole” in combination with liquid potassium soaps), halogenated (chlorinated, brominated) phenols, such as hexachlorophene, triclosan, trichlorophenol, tribromophenol, pentachlorophenol, DIBROMOL and salts thereof), cationic surfactants, such as some quaternary ammonium cations (such as benzalkonium chloride, cetyl trimethylammonium bromide or chloride, didecyldimethylammonium chloride, cetylpyridinium chloride, benzethonium chloride) and others, non-quarternary compounds, such as chlorhexidine, glucoprotamine, octenidine dihydrochloride etc.) strong oxidizers, such as ozone and permanganate solutions; heavy metals and their salts, such as colloidal silver, silver nitrate, mercury chloride, phenylmercury salts, copper sulfate, copper oxide-chloride, strong acids (phosphoric, nitric, sulfuric, amidosulfuric, toluenesulfonic acids) and alkalis (sodium, potassium, calcium hydroxides).

Other such agents include properly diluted chlorine preparations (Daquin's solution, 0.5% sodium or potassium hypochlorite solution, sodium benzenesulfochloramide (chloramine B)), some iodine preparations, such as iodopovidone in various galenics (ointment, solutions, wound plasters), peroxides as urea perhydrate solutions and pH-buffered 0.1-0.25% peracetic acid solutions, alcohols, sorbic acid, benzoic acid, lactic acid and salicylic acid, hexachlorophene, triclosan and DIBROMOL, and cation-active compounds, such as 0.05-0.5% benzalkonium, 0.5-4% chlorhexidine, 0.1-2% octenidine solutions.

Other such agents include ephalosporins, β-lactam antibiotics, aminoglycosidic antibiotics, fluoroquinolones, nitrofurans, vancomycin, monobactams, co-trimoxazole, and metronidazole. Anti-fungal/antibacterial agents used in treating hot tub water to minimize the waterline formed between the wall of the hot tub and the surface of the water can be used. Such agents can be found in U.S. Pat. Nos. 5,668,084 and 5,449,658, the relevant parts of which are hereby incorporated by reference. These agents can include, for example, polyhexamethylene, biguanide compound, a water-soluble or water-dispersible surfactant, di(hydrogenated tallow alkyl)-dimethyl quaternary ammonium chloride, tallow alkyl benzyl dimethyl quaternary ammonium chloride, hydrogenated tallow alkyl benzyl dimethyl quaternary ammonium chloride, di(hydrogenated tallow alkyl)-dimethyl quaternary ammonium chloride, tallow alkyl benzyl dimethyl quaternary ammonium chloride and hydrogenated tallow alkyl benzyl dimethyl quaternary ammonium chloride, alkoxylated alkanolamides.

Other such agents include polyoxypropylene block copolymers, ethoxylated propoxylated alcohols, fatty alcohol polyglycol ethers, alkylene oxide addition products, methyl bis(soya alkyl amidoethyl) 2-hydroxyethyl quaternary ammonium methyl sulfate and methyl bis(tallow alkyl amidoethyl) 2-hydroxyethyl quaternary ammonium methyl sulfate, alkoxylated alkanolamides, poly(hexamethylene biguanide) hydrochloride (PHMB), ethylenediamine-tetraacetic acid or a salt thereof (EDTA) as a calcium ion-chelating agent.

Other such agents include a polyhexamethylene, biguanide compound and a water-soluble or water-dispersible surfactant selected from the group consisting of di(hydrogenated tallow alkyl)-dimethyl quaternary ammonium chloride, tallow alkyl benzyl dimethyl quaternary ammonium chloride and hydrogenated tallow alkyl benzyl dimethyl quaternary ammonium chloride,

One of skill can select a therapeutically effective concentration of any desired anti-infective agent. In some embodiments, for example, ciclopirox may be used in concentrations ranging from about 0.1% to about 10.0%, from about 1.0% to about 8.0%, from about 2.0% to about 6.0%, from about 3.0% to about 5.0%, from about 5.0% to about 9.0%, or any range therein. It should be appreciated that the selection of the carrier medium for the anti-infective agent, the selection of the anti-infective agent, as well as the selection of the time, temperature, and cycling regime will affect the concentration desired in any given treatment program.

Any carrier medium for the anti-infective agents known to one of skill that to be considered suitable for purposes of the present invention can be used. In some embodiments, aqueous carrier mediums may be preferred. In some embodiments, for example, the anti-infective agent is administered in a pharmaceutically acceptable carrier for topical administration. The pharmaceutically acceptable topical carrier, for example, may comprise one of a number of known acceptable forms including known aqueous carriers and oleaginous carriers. In some embodiments, the carrier can be a conventional carrier, such as, a cream, ointment, solution or gel containing appropriate stabilizers, buffers and preservatives for topical application. Hydrophilic or hydrophobic ointments may be employed as carriers in some embodiments. It should be appreciated, however, that hydrophobic ointments, such as VASELINE, which are based upon hydrocarbon and wax derivatives may not be as efficacious as the hydrophilic ointments because they may tend to inhibit penetration through the nail, whereas hydrophilic ointments such as those based upon propylene glycol, polyalkylene glycols, and pluronics may tend to enhance penetration.

The carriers can include complementary agents. For example, one of skill may use a keratolytic agent to pretreat the nail, and a mixture of a keratolytic agent and anti-infective agent can be applied to the nail and heated. In some embodiments, for example, the nail can be pretreated in 17% salicylic acid, and a mixture of 10 ml of 1% ciclopirox and 5 ml of 1% salicylic acid in 120 ml water is thickened with a thickener and applied to the dorsal surface of the nail and heated.

The agents may be diluted and thickened into a paste using any thickener known to one of skill to be useful and acceptable for the applications taught herein. The thickening agents can be selected, for example, from the group consisting of pectin, corn starch, acetylated distarch adipate, agar, alginic acid, arrowroot, beta-glucan, beurre manié, calabash nutmeg, calcium alginate, carrageenan, cassia gum, chondrin, collagen, dextrin, e1100, erythronium japonicum, fecula, filé powder, galactomannan, gelatin, gellan gum, glucomannan, guar gum, gum karaya, irvingia, konjac, kudzu, locust bean gum, methylcellulose, millet jelly, modified starch, natural gum, njangsa, pearl sago, pearl tapioca, phosphated distarch phosphate, potassium alginate, psyllium seed husks, roux, sago, salep, starch, tapioca, tragacanth, waxy corn, xanthan gum, polyethylene glycol, polyacrylic acid, vegetable gums, petroleum jelly, flour, and combinations thereof.

The anti-infective agent can also comprise a surfactant to assist in controlling the surface tension between the keratin of the nail and the anti-infective agent delivery medium. In some embodiments, the surface tension is lowered using the surfactant. The surfactant can be, for example, anionic, cationic, amphoteric, or even non-ionic, wherein the surfactant can be selected to complement any delivery medium of the anti-infective agent. The surfactant can be selected and added in the concentration desired. In some embodiments, the surfactant is combined with a carrier in a concentration range of between approximately 0.5% to 2%, by weight. Other concentrations are acceptable and useful, as the surfactant may be present in a concentration of between approximately 0.1% to 10% by weight in some embodiments. More than one surfactant may be selected and combined with any formulation provided herein. For example, the selected surfactants may also be combined in varying concentration ranges including, for example, between approximately 0.1% to 20% by weight, between approximately 0.05% to 50% by weight, between approximately 1.0% and 10% by weight and between approximately 0.5% and 5.0% by weight.

The teachings are also directed to a method of treating an infected nail of a subject. In some embodiments, the methods can comprise obtaining a system described above, for example, and directly applying the anti-infective agent to the dorsal surface of the infected nail in the absence of a nail-infection-agent-containing member or sponge for receiving and delivering the anti-infective agent to the infected nail. The methods can further comprise releasably attaching the holding mechanism to the digit having the infected nail, wherein the releasably attaching includes directly contacting the anti-infective agent applied to the dorsal surface of the infected nail with the surface of the heating element. Heat is applied to the anti-infective agent contact is maintained between the anti-infective agent and the heating element to heat the anti-infective agent for a therapeutically effective treatment time.

As described above, the application of heat facilitates the transport of the anti-infective agent through the infected nail. As such, in some embodiments, the heating element maintains a temperature of up to about 45° C. at the dorsal surface of the nail. And, in some embodiments, the temperature gradient across the infected nail ranges from up to about 45° C. at the dorsal surface of the nail to about 37° C. or less at the plantar or palmar surface of the digit.

In some embodiments, the above-described chemistries can be used. However in some embodiments, the applying of the anti-infective agent to the dorsal surface of the infected nail comprises first applying a keratolytic agent without the application of heat as a pretreatment and then applying an anti-infective agent with heat. In these embodiments, for example, the keratolytic agent can be undecylenic acid, and the anti-infective agent can be terbinafine or clotrimazole.

FIG. 3 illustrates an apparatus that directly applies an anti-infective agent to the dorsal surface of an infected nail in the absence of a nail-infection-agent-containing member or sponge, according to some embodiments. The apparatus 300 can comprise a heating element 305 having a surface 310 adapted for a direct contact with an anti-infective agent 315 administered directly to a dorsal surface 320 of an infected nail 325 of a digit 330 of a foot 335 of a subject. The surface 320 can be adapted for any of a variety of functional purposes such as, for example, to provide additional comfort, or for inhibiting a leakage of the anti-infective agent 315 from the drug delivery mechanism. The apparatus can also comprise a holding mechanism 340 for releasably attaching the heating element 305 to the digit 330 having the infected nail 325 and maintaining the direct contact between the heating element 305 and the anti-infective agent 315 as administered directly to the dorsal surface 320 of the infected nail 325. Heating element 305 is powered by power sources 360, 370 and is connected to the power sources by electrical wires 380. In these embodiments, however, the apparatus 300 does not comprise a nail-infection-agent-containing member or sponge for receiving and delivering the anti-infective agent 315 to the infected nail; the holding mechanism 340 comprises a substantially open structure that covers the infected nail 325 and does not enclose the digit 330; and, the heating element 305 functions to heat the anti-infective agent 315 to a temperature greater than about 40° C. and facilitate an enhanced trans-keratin drug delivery of the anti-infective agent 315 through the infected nail 325 in a dark, warm, and moist environment that prevents the growth of fungi in the infected nail 325.

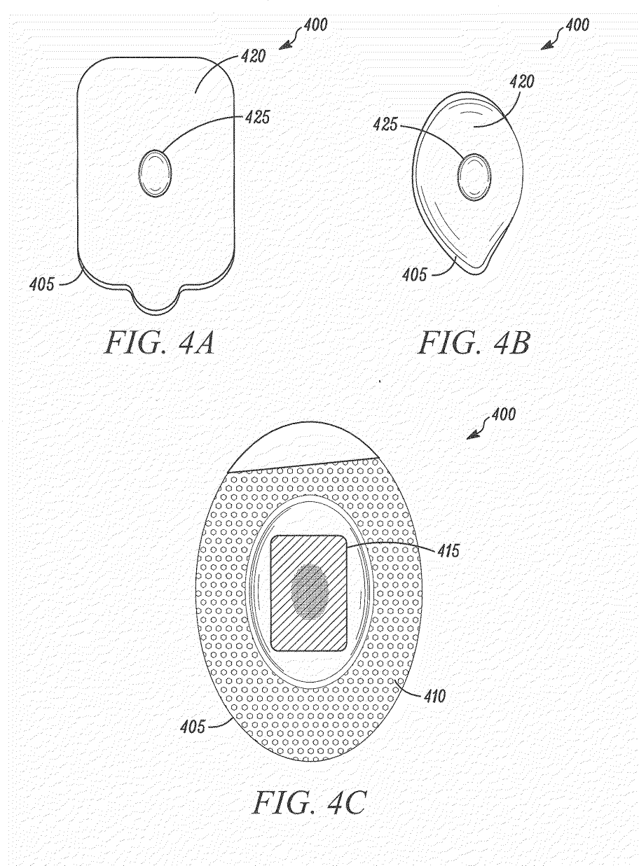

The teachings are also directed to components that can be used for treating an infected nail using the teachings provided herein. FIGS. 4A-4C illustrate a heatable bandage for treating an infected nail, according to some embodiments. The heatable bandage 400 can comprise, for example, a conformable sheet of material 405, which can comprise a woven or non-woven cloth material, a plastic material, or an otherwise formed sheet of material. It should be appreciated that the material can be of virtually any practical thickness, as long as the material can conform to the digit, hold to the digit, and retain the anti-infective agent.

In some embodiments, the material is designed to have an adhesive side 410 to contact and adhere to the digit, house a heating element 415, and directly contact and heat the anti-infective agent with the heating element 415. In some embodiments, the bandage holds to the digit by a mechanism other than adhesion such as, for example, a hook and loop Velcro, a latch strap mechanism, or some other mechanical mechanism known to one of skill. The material also has an external side 420 that is exposed to the external environment and shields the anti-infective agent from the external environment to retain the anti-infective agent in contact with the dorsal surface of the infected nail.

In some embodiments, the sheet of material 405 can be designed, for example, to hold a heating element 415 against an anti-infective agent applied to a dorsal surface of an infected nail under most any condition, such as in an open air environment, a closed environment of a shoe or glove, or perhaps even a moist environment, for example, to retain the anti-infective agent in heated contact with the dorsal surface of the infected nail. In these embodiments, the heatable bandage 400 can contain an electrical connection 425 for operably connecting a power source to the heating element 415. The adhesive side of the sheet of material 405 can be designed to adhere to epidermal tissues of a digit that surround an infected nail. The heating element 415 can be designed to heat the anti-infective agent to a temperature greater than about 40° C., for example, and facilitate an enhanced trans-keratin drug delivery of the anti-infective agent through the infected nail in a dark, warm, and moist environment that prevents the growth of fungi in the infected nail. In some embodiments, the system can include the heatable bandage 400, a power source, and an anti-infective agent.

Without intending to be limited to any theory or mechanism of action, the following examples are provided to further illustrate the teachings presented herein. It should be appreciated that there are several variations contemplated within the skill in the art, and that the examples are not intended to be construed as providing limitations to the claims.

### Example 1

A subject having an onychomycosis diagnostic of a Trichophyton genus was treated using a variety of the methods taught herein, generally according to a method taught by U.S. application Ser. No. 11/423,874, which is hereby incorporated herein by reference in its entirety. The presence of fungal hyphae in the subject was consistent with Trichophyton Rubrum or Trichophyton Mentagrophytes. Nails infected with these organisms also produce a chronic type of tinea pedis. Infections involving the interdigital areas can produce erythema, fissuring, and may extend into other portions of the foot.

In this example, The infected nail was submerged in a warm salicylic acid and ciclopirox solution at a temperature ranging from about 43° C. to about 46° C. for 30 to 45 minutes daily. The solution was made by dissolving 5.0 ml of 1% LOPROX (ciclopirox) shampoo and 5.0 ml of 3% DERMAREST psoriasis (salicylic acid) shampoo in 120 ml of warm water. Approximately 5.0 ml of this mixture was applied to a sponge, and the entire device was heated in a 1100 watt microwave for 15 seconds to obtain the temperature range above. The delivery system was wrapped around the infected toenail(s) for a period of 75 days, for 20 minutes twice daily.

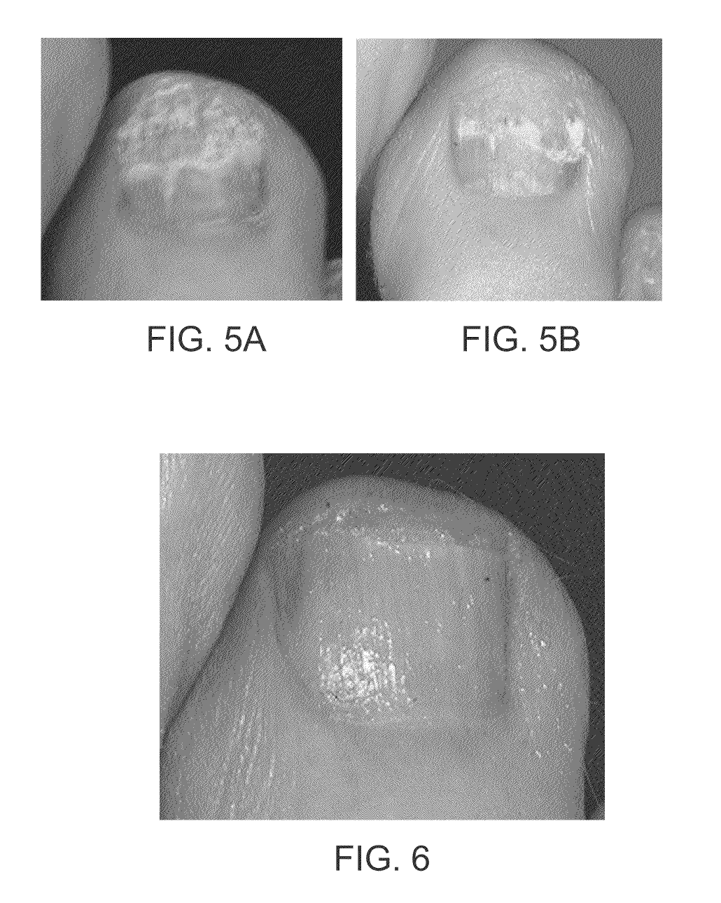

FIGS. 5A and 5B show the results of a treatment using an anti-infective agent retained by a sponge, heated, and applied to the dorsal surface of an infected nail, according to some embodiments. FIG. 5A shows the nail before treatment, and FIG. 5B shows the nail after treatment. The use of a heated sponge to soak the infected nail in heated keratolytic and antifungal solutions improved the appearance of the nail. However, the treatment failed to cure the condition either clinically or mycologically.

### Example 2

The subject of example 1 was treated by applying the anti-infective agent directly to the dorsal surface of the nail, without using a sponge to hold the agent, and then directly heating the anti-infective agent by directly contacting the agent with a heating element.

In this example, a ciclopirox L0.77% solution was applied topically to the infected nail twice daily. After this application, the heating device shown in FIG. 3 was used to heat the infected nail and ciclopirox solution to about 41 C for 30 minutes twice daily for a period of 90 days.

| FIG Label | | |
|---|---|---|
| No. | Component | Parts |
| 305 | Heating Element | Foam Sheet; 12 VDC Silicone |
| 310 | | Heater; Velcro Hook & Loop PSA |
| 340 | Holding Mechanism for | Buna O-Ring AS568A-222; Buna |
| | Heating Element | O-Ring AS568A-275; |
| | | Polyethylene Insulation |
| 360 | Power Source for Heater | 2-3X NIMH AA Batteries; Battery |
| 370 | and Connector Wires | Holder (2 cells); Battery Holder (1 |
| 380 | | cell); Wire-Two Conductor; Velcro |
| | | Hook 2″ Wide PSA; Velcro Loop |
| | | 2″ Wide Plain; Nylon Cable Tie 4″ |
| | | Long; Velcro Hook & Loop PSA; |
| | | Leviton Cord Switch; 9 Volt Snap |
| | | Connector |

FIG. 6 shows the successful treatment of an infected nail using the teachings described herein, according to some embodiments. The results shown in FIG. 6 suggest that by heating the medication applied directly to the infected nail, instead of heating the agent and applying it indirectly from a sponge, improves the absorption of the medication through the nail. As can be seen from FIG. 6, this treatment eliminates the infection and provides the clinical cure.

We claim:

1. A system for treating an infected nail, comprising:
a drug delivery mechanism comprising an anti-infective agent and a heating element having a surface adapted for a direct contact with the anti-infective agent as administered directly to a dorsal surface of an infected nail of a digit of a subject; and,
a holding mechanism for releasably attaching the drug delivery mechanism to the digit having the infected nail and maintaining the direct contact between the heating element and the anti-infective agent as administered directly to the dorsal surface of the infected nail;
wherein,
the drug delivery mechanism does not comprise a nail-infection-agent-containing member or sponge for receiving and delivering the anti-infective agent to the infected nail;
the holding mechanism comprises a substantially open structure that covers the infected nail and does not enclose the digit; and,
the system facilitates an enhanced trans-keratin drug delivery of the anti-infective agent through the infected nail in a dark, warm, and moist environment that prevents the growth of fungi in the infected nail.

2. The system of claim 1, wherein the holding mechanism promotes a convection of heat in the direction of the nail bed to facilitate an enhanced trans-keratin drug delivery from the dorsal surface of the nail to the nail bed, the holding mechanism providing substantially more heat insulation over the dorsal surface of the nail than a plantar or palmar surface of the digit to promote convection of heat toward the nail bed of the digit.

3. The system of claim 1, wherein the surface of the heater is adapted for a direct contact with the anti-infective agent and comprises a non-polar material having a contact angle of over 90 degrees with the anti-infective agent.

4. The system of claim 1, wherein the heating element maintains a temperature of about 45° C. at the dorsal surface of the nail.

5. The system of claim 1, wherein the temperature gradient across the digit having the infected nail ranges from about 45° C. at the dorsal surface of the nail to about 37° C. or less at the plantar or palmar surface of the digit.

6. The system of claim 1, wherein the anti-infective agent comprises a keratolytic agent selected from the group consisting of lactic acid, allantoin, zinc pyrithione, sulfur, rosorcinol, undecylenic acid, and combinations thereof.

7. The system of claim 1, wherein the anti-infective agent comprises a surfactant.

8. The system of claim 1, wherein the anti-infective agent comprises an antifungal agent selected from the group consisting of clotrimazole, ketoconazole, miconazole, butenafine, econazole, lotrisone, naftifine, nystatin, oxiconazole, sulconazole, terbinafine, tolnaftate, sertaconazole, ertaczo, undecylenic acid, and combinations thereof.

9. The system of claim 1, wherein the anti-infective agent comprises a component that functions as an antifungal, antibacterial, surfactant, keratolytic, and drying agent.

10. The system of claim 9, wherein the anti-infective agent comprises a component selected from the group consisting of hydrogen peroxide, poly(iminoimido carbonyl iminoimido carbony liminohexamethylenehydrochloride), bis(hydrogenated tallow alkyl) dimethyl quarternary chlorides, bis(hydrogenated alkyl methyl amines) hydrochloride, and combinations thereof.

11. The system of claim 9, wherein the anti-infective agent is selected from the group consisting of 1-bromo-3-chloro-5-dimethylhydantoin, 1,3-dichloro-5-dimethylhydantoin, 1,3-dichloro-5-ethyl-5-methylhydantoin, potassium peroxymonosulfate, and sodium dichloro-s-triazinetrione dehydrate.

12. A method of treating an infected nail of a subject, wherein the method comprises:
obtaining the system of claim 1;
directly applying the anti-infective agent to the dorsal surface of the infected nail in the absence of a nail-infection-agent-containing member or sponge for receiving and delivering the anti-infective agent to the infected nail;
releasably attaching the holding mechanism to the digit having the infected nail, wherein the releasably attaching includes directly contacting the anti-infective agent applied to the dorsal surface of the infected nail with the surface of the heating element;
applying heat to the anti-infective agent; and,
maintaining contact between the anti-infective agent and the heating element to heat the anti-infective agent for a therapeutically effective treatment time.

13. The method of claim 12, wherein the heating element maintains a temperature of about 45° C. at the dorsal surface of the nail.

14. The method of claim 12, wherein the temperature gradient across the infected nail ranges from about 45° C. at the dorsal surface of the nail to about 37° C. or less at the plantar or palmar surface of the digit.

15. The method of claim 12, wherein the anti-infective agent comprises a keratolytic agent.

16. The method of claim 15, wherein the anti-infective agent comprises a keratolytic agent selected from the group consisting of lactic acid, allantoin, zinc pyrithione, sulfur, rosorcinol, undecylenic acid, and combinations thereof.

17. The method of claim 12, wherein the anti-infective agent comprises a surfactant.

18. The method of claim 12, wherein the anti-infective agent comprises an antifungal agent selected from the group consisting of clotrimazole, ketoconazole, miconazole, butenafine, econazole, lotrisone, naftifine, nystatin, oxiconazole, sulconazole, terbinafine, tolnaftate, sertaconazole, ertaczo, undecylenic acid, and combinations thereof.

19. The method of claim 12, wherein the anti-infective agent comprises a component that functions as an antifungal, antibacterial, surfactant, keratolytic, and drying agent.

20. The method of claim 19, wherein the anti-infective agent comprises a component selected from the group consisting of hydrogen peroxide, poly(iminoimido carbonyl iminoimido carbony liminohexamethylenehydrochloride), bis(hydrogenated tallow alkyl) dimethyl quarternary chlorides, bis(hydrogenated alkyl methyl amines) hydrochloride, and combinations thereof.

21. The method of claim 19, wherein the anti-infective agent comprises a component selected from the group consisting of 1-bromo-3-chloro-5-dimethylhydantoin, 1,3-dichloro-5-dimethylhydantoin, 1,3-dichloro-5-ethyl-5-methylhydantoin, potassium peroxymonosulfate, and sodium dichloro-s-triazinetrione dehydrate.

22. The method of claim 12, wherein the applying the anti-infective agent to the dorsal surface of the infected nail comprises first applying a keratolytic agent without the application of heat as a pretreatment and then applying an anti-infective agent with heat.

23. The method of claim 22, wherein the keratolytic agent is undecylenic acid, and the anti-infective agent is terbinafine or clotrimazole.

24. An apparatus for treating an infected nail, comprising:
a heating element having a surface adapted for a direct contact with an anti-infective agent as administered directly to a dorsal surface of an infected nail of a digit of a subject, wherein the surface is adapted for inhibiting a leakage of the anti-infective agent from the drug delivery mechanism; and,
a holding mechanism for releasably attaching the heating element to the digit having the infected nail and maintaining the direct contact between the heating element and the anti-infective agent as administered directly to the dorsal surface of the infected nail;
wherein,
the apparatus does not comprise a nail-infection-agent-containing member or sponge for receiving and delivering the anti-infective agent to the infected nail;
the holding mechanism comprises a substantially open structure that covers the infected nail and does not enclose the digit; and,
the heating element functions to heat the anti-infective agent to a temperature greater than about 40° C. and facilitate an enhanced trans-keratin drug delivery of the anti-infective agent through the infected nail in a dark, warm, and moist environment that prevents the growth of fungi in the infected nail.

25. A heatable bandage for treating an infected nail, comprising:
a conformable sheet of material having an adhesive side and an external side, wherein the sheet of material functions to hold a heating element against an anti-infective agent applied to a dorsal surface of an infected nail; and
an electrical connection for operably connecting a power source to the heating element;
wherein:
the adhesive side adheres to epidermal tissues of a digit that surround an infected nail; and,
the heating element functions to heat the anti-infective agent to a temperature greater than about 40° C. and facilitate an enhanced trans-keratin drug delivery of the anti-infective agent through the infected nail in a dark, warm, and moist environment that prevents the growth of fungi in the infected nail.

26. A system comprising the heatable bandage of claim 25 and an external power source for heating the anti-infective agent.
